(19) United States
(12) Patent Application Publication
(10) Pub. No.: US 2025/0152934 A1
(43) Pub. Date: May 15, 2025

(54) System and Method for Grounding Humans to Prevent Aerosolization of Lung Pathogens

(71) Applicant: Steven Mark Hayden, Reno, NV (US)

(72) Inventor: Steven Mark Hayden, Reno, NV (US)

(21) Appl. No.: 18/509,068

(22) Filed: Nov. 14, 2023

(51) Int. Cl. A61N 1/14; A43B 7/36; H05K 9/00

(52) U.S. Cl. CPC A61N 1/14; H05K 9/0064; H05K 9/0067; A43B 7/36

(57) ABSTRACT

The present invention is a method and system for storing charge on a body and subsequently electrically grounding that body so the body can operate without the unwanted effects of an electrical charge. The system of the present invention comprises an electrical conducting medium, which may be a human body, a capacitance article, and a grounding apparatus. In use the capacitance article is connected to the electrical conducting medium to reduce the buildup of electrical charge on the electrical conducting medium and instead store the charge in the capacitor of the capacitance article. The electrical conducting medium may then be connected to the grounding apparatus at a predetermined interval remove any built-up charge on both the capacitance article and the electrical conducting medium by putting those components in electrical continuity with the ground.

### FIELD OF THE INVENTION

The present invention relates generally to methods and apparatuses for reducing electric charge on a body and specifically reducing electric charge through grounding to prevent aerosolization of airway particles.

### BACKGROUND OF THE INVENTION

Viruses infect all forms of life, from humans to plants to bacteria. In humans and animals, many viruses, such as influenza, SARS-CoV-2, and measles, can spread through the airborne transmission. Airborne transmission of an infectious disease from a person may occur through breathing, talking, coughing, sneezing, or any other action that generates and expels aerosol particles or droplets.

Particulate matter breathed in by a person may be filtered out in the upper airways, but many of the particles make their way to the lungs and rest on the alveolar wall. Conversely, particles exhaled from a person may be filtered out in the upper airways, but many are exhaled completely. When a particle is charged oppositely a person, it behaves differently than with an uncharged body. The particle is repelled from both the walls of the airways and from the alveolar surface, making the intake and output of particles more efficient. Any minor vibration of the alveolar wall through talking, singing, or otherwise making noise may be sufficient to remove a viral particle from the alveolus and send it with exhaled air out the airways. Since charge effects objects with smaller mass more, a particle being exhaled from a person may be significantly affected by the charge on the person due to the small size of the particle, even if the charge is unnoticeable to the much larger person.

Electrostatic charge can build up on a person through regular activity. This generally occurs when certain materials rub against one another. Materials such as wool, fur, glass, human hair, and nylon tend to lose electrons when in contact with other materials, while materials such as rubber, silicone, and plastic tend to gain electrons when in contact with other materials. Thus, when a material that tends to lose electrons rubs against a material that tends to gain electrons, electrons are transferred, and each material gains an opposite charge. For example, a person walking on a vinyl floor with rubber-soled shoes may become negatively charged or a person brushing against something plastic while wearing a wool sweater may become positively charged. In situations such as these, the person may stay charged and build up additional charge until grounded. This electrical charge carried on the body can lead to a painful and unexpected shock during discharge as well as unwanted effects on particles in the body.

The spread of disease by aerosolization of charged particles is a significant issue caused in part by carrying unwanted and unnecessary charge. One example of the unwanted spread of particles is in stadiums. The plastic seats commonly used in stadiums may cause a charge to generate on people. This charge in combination with the yelling/cheering that often occurs in stadiums can lead to increased aerosolization of virus particles in a crowded space. Another similar situation is in churches, where the prevalence of wood keeps people from becoming grounded and the regular singing can lead to increased aerosolization among those who have developed a charge on their person, all in a crowded space. In addition to these examples, many other common areas exist that cause people to develop a charge on their body while limiting natural grounding.

Many steps have been taken to reduce the spread of disease. Vaccines can aid in preventing people from getting sick when exposed to a virus and face masks can limit the spread of particulate matter that is aerosolized and expelled from a person. However, there exists a further need for methods and systems to reduce the charge on person's body to limit aerosolization of lung pathogens and stop their spread at the source.

### SUMMARY OF THE INVENTION

The present invention is a method and apparatuses for electrically grounding a body to prevent the aerosolization of lung pathogens. The system of the present invention comprises a capacitance article, a grounding apparatus, a body or other electrical conducting medium, and an electrical ground. The capacitance article of the present invention comprises a capacitor that can be attached to the body by an attachment portion. The grounding apparatus of the present invention comprises a conductive surface and a grounding connector that connects the conducting surface to the electrical ground. The grounding apparatus may also include a switch and a resistor as additional control elements. The method of the present invention comprises attaching the capacitance article to the body so charge that would otherwise build up on the body may build up in the capacitor instead. The grounding apparatus is then connected to the electrical ground and the body put in contact with the conductive surface of the grounding apparatus to remove excess charge from the capacitor and the body.

### BRIEF DESCRIPTION OF THE DRAWINGS

FIG. 1 is an illustration of the capacitance article of the present invention in accordance with at least one embodiment.

FIG. 2 is an illustration of the capacitance article of the present invention in accordance with at least one embodiment.

FIG. 3 is an illustration of the capacitance article of the present invention in accordance with at least one embodiment.

FIG. 4 is a diagram of the capacitor of the present invention in accordance with at least one embodiment.

FIG. 5 is an illustration of the grounding device of the present invention in accordance with at least one embodiment.

FIG. 6 is an illustration of the grounding device of the present invention in accordance with at least one embodiment.

FIG. 7 is an illustration of the grounding device of the present invention in accordance with at least one embodiment.

FIG. 8 is a diagram of the system of the present invention in accordance with at least one embodiment.

FIG. 9 is circuit diagram of the present invention in accordance with at least one embodiment.

FIG. 10 is a flowchart of the method of the present invention in accordance with at least one embodiment.

FIG. 11 is a flowchart of the method of the present invention in accordance with at least one embodiment.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

All illustrations of the drawings are for the purpose of describing selected versions of the present invention and are not intended to limit the scope of the present invention.

It is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings. Accordingly, the following detailed description does not limit the disclosure. Instead, the proper scope of the disclosure is defined by the appended claims.

Particles are regularly inhaled and exhaled to and from the air by people throughout their daily lives. These particles are inclusive of virus particles, but also include mineral dust, gasses, and other organic matter. Viral particles regularly rest on a person's alveolar membrane in their lungs. However, these particles may pop off of the alveolar membrane through vibration and be exhaled, spreading the virus through the air. This spread is significantly increased when a person's body has an electrical charge opposite that of the particles. In these situations, the charged particles are easily removed from the alveolar membrane due to the opposing charge, and easily travel through the upper airways as they are repelled from the airway walls.

The present invention is a method and system for electrically grounding a person in order to reduce the aerosolization of lung pathogens. The present invention comprises an electrical conducting medium 3, a capacitance article 1, and a grounding apparatus 2. The capacitance article may further comprise a capacitor 11 and an attachment portion 12. The grounding apparatus may further comprise a conductive surface 21, a grounding connector 22, a resistor 23, and a switch 24. The method of the present invention generally comprises connecting a capacitance article 1 to the electrical conducting medium 3 of a user and subsequently grounding the capacitance article 1 by using a grounding apparatus 2 to create electrical continuity with the ground. The capacitance article 1 of the present invention is worn on the electrical conducting medium 3 to collect charge picked up by the user. The grounding apparatus 2 of the present invention is used to create electrical continuity between the user and the ground in a manner that is safe for the user.

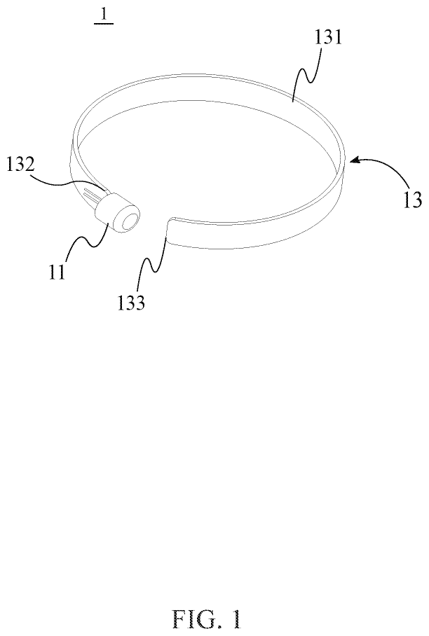

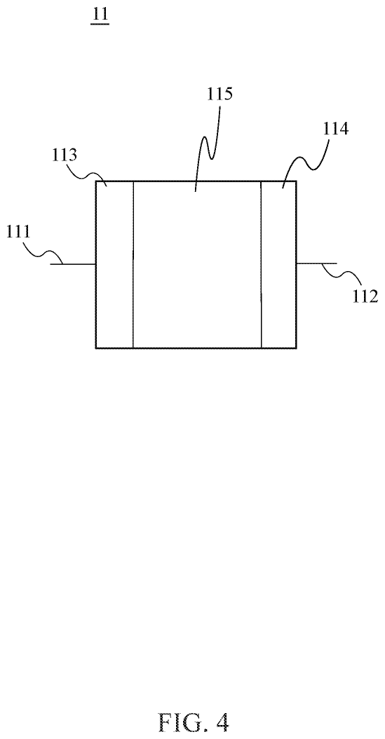

As shown in FIG. 1-3, The capacitance article 1 of the present invention comprises a capacitor 11 and an attachment portion 12. In one embodiment, shown in FIG. 4, the capacitor 11 is a generic capacitor 11 comprising a first terminal 111, a second terminal 112, a first conducting plate 113, a second conducting plate 114, and an insulating dielectric 115. The elements of the capacitor 11 are arranged with the first conducting plate 113 and the second conducting plate 114 separated by and in direct contact with the insulating dielectric 115. The first terminal 111 is attached to the first conducting plate 113 and the second terminal 112 is attached to the second conducting plate 114, with neither the first terminal 111 nor the second terminal 112 being in contact with the insulating dielectric 115. In some embodiments, an insulating coating 116 covers the first conducting plate 113, second conducting plate 114, and insulating dielectric 115, while leaving the first terminal 111 and the second terminal 112 protruding from the capacitor.

The attachment portion 12 of the capacitance article 1 is a string, band, clip, garment, or other mechanism that holds the capacitor 11 against the electrical conducting medium 3 or puts the capacitor 11 in electrical contact with the electrical conducting medium 3. In the preferred embodiment, shown in FIG. 1, the attachment portion 12 is a band of copper, sized to be worn around the user's wrist. In this embodiment, the band is preferably horseshoe 15 shaped, having a curved body with two open ends, such that the circumference of the band can be slightly increased by pulling on the two open ends and securely placed around the user's wrist. The capacitor 11 may be attached to the band in this embodiment in a variety of ways, with the preferred attachment being securing the first terminal 111 and second terminal 112 of the capacitor 11 to one of the open ends of the band. In alternative embodiments, the attachment portion 12 of the capacitance article 1 may be a conductive chain to be worn around the user's neck or any other conductive material worn against the user's skin. In some embodiments, the capacitor 11 may be sewn into or otherwise incorporated into an article of clothing such as a shirt, hat, or shoe 15 and positioned such that it is regularly in electrical continuity with the user's electrical conducting medium 3. This may mean positioning the capacitor 11 in the article of clothing so that the first terminal 111 and second terminal 112 stay in regular contact with the electrical conducting medium 3 or using a conductive material in the article of clothing so that the capacitor 11 is electrically connected to the electrical conducting medium 3 even if it in not in direct physical contact.

The electrical conducting medium 3 in the preferred embodiment is the body of the user. The capacitance article 1 of the present invention allows the user to go about their daily life without significant buildup of electric charge on the electrical conducting medium 3. By wearing one or more capacitance articles 1, the charge built up during daily activity can be primarily stored in the capacitance articles 1 rather than on the electrical conducting medium 3. This storage of excess charge in the capacitance articles 1 specifically reduces the charge on the user's alveolus, minimizing the aerosolization of the particles on the alveolar surface through breathing, talking, singing, etc. While the capacitance articles 1 of the present invention can reduce some charge on the electrical conducting medium 3, the storage capacity of the capacitance articles 1 is limited. This limit is governed by the number of capacitance articles 1 a user is willing to wear, as well as the danger of building up significant charge in the capacitance article 1 and potentially injuring the user or others through unplanned discharge. Thus, the capacitance articles 1 of the present invention must be discharged regularly, preferably through the controlled use of the grounding apparatus 2 of the present invention.

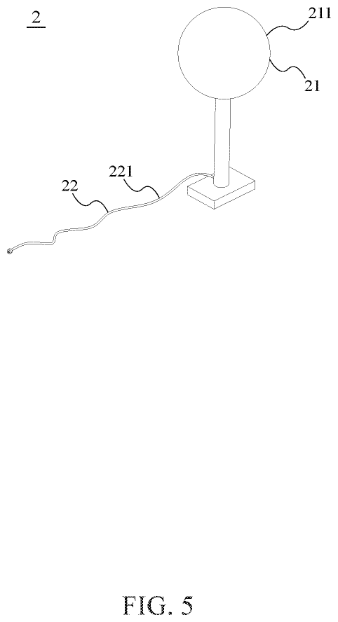

As shown in FIG. 5-7, the grounding apparatus 2 of the present invention may be any conductive, grounded surface that allows for electrical continuity between the user and the ground when physical contact is made between the user and the grounding apparatus. In most embodiments, the grounding apparatus 2 of the present invention comprises a conductive surface 21, a grounding connector 22, and a resistor 23. The conductive surface 21 of the grounding apparatus 2 is a conductive piece of material able to be contacted by the electrical conducting medium 3, preferably over a large surface area. The conductive surface 21 may be, but is not limited to, a sheet, a handle, and a sphere, each made entirely of a conducting material. In the preferred embodiment, the electrical conducting medium 3 contacts the conductive surface 21 by the user placing th entirety of their palm on the conductive surface 21. In alternative embodiments, any part of the electrical conducting medium 3 may contact the conductive surface 21 with, such as by standing on the conductive surface 21 with bare feet or placing another electrical conducting medium 3 atop the conductive surface 21. Additionally, the capacitance article 1 may be directly discharged by putting the capacitor 11 in electrical continuity with the conductive surface 21. While the conductive surface 21 may be contacted by the electrical conducting medium 3 at a single point, such as the tip of the user's finger, a larger area of contact between the user and the conductive surface 21 allows the discharge to occur over a greater area, protecting the electrical conducting medium 3 and reducing painful shocks in cases where the electrical conducting medium 3 is a person's body.

The grounding connector 22 of the grounding apparatus 2 is a conductive material that connects the conductive surface 21 to the electrical ground 4, allowing electric charge on the conductive surface 21 to dissipate to the earth. In its preferred embodiment, the grounding connector 22 is a wire with a first end and a second end. The first end of the wire is connected to the conductive surface 21 and the second end of the wire is connected to the ground port of an electrical outlet. In some embodiments, the second end of the wire may be connected to a ground plug adapter to easily connect the grounding connector 22 to a common outlet. The ground plug adapter in its preferred embodiment has one inlet port, two insulating prongs, and one grounding prong. The inlet port of the ground plug receives the grounding connector 22, the two insulating prongs are inserted into the contact openings on the outlet to secure the ground plug adapter in the outlet, and the grounding prong is inserted into the ground opening on the outlet to connect the grounding connector 22 to the electrical ground 4.

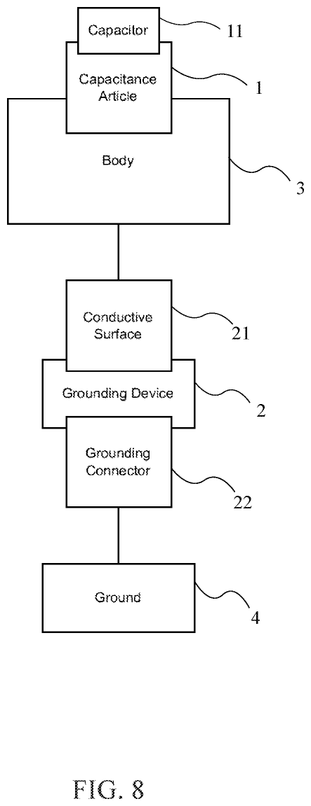

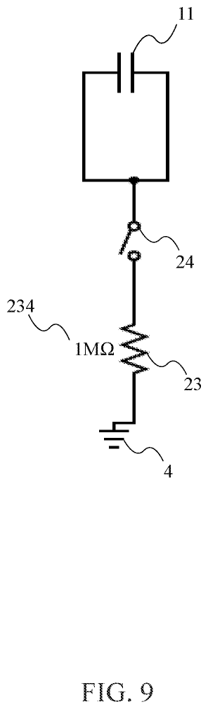

One embodiment of the physical and electrical connections of the components is shown in FIG. 8-9. The resistor 23 of the grounding apparatus 2 in most embodiments is a common resistor 23. The resistor 23 comprises a first lead 231, a second lead 232, and a resistor body 233. The resistor 23 may be place at any point between the conductive surface 21 and the ground. In the preferred embodiment, the first lead 231 of the resistor 23 is connected to the grounding connector 22 and the second lead 232 of the resistor 23 is connected to the ground 4. The resistor body 233 is generally comprised of a mixture of carbon and ceramic in the amounts necessary to reach the desired resistance level. The resistance value 234 of the resistor 23 may vary across embodiments of the grounding apparatus. In the preferred embodiment, the resistance value 234 of the resistor 23 is approximately one megaohm. This level of resistance generally allows charge to dissipate in less than a second without causing pain to the user. A shock to the user is more likely to be noticeable at lower resistances and the time to dissipate the charge will be longer at higher resistances. Thus, in alternative embodiments, higher or lower resistances may be used if the user determines that discharge speed or avoiding shock is of particular importance in the user's specific situation.

In some embodiments, the grounding apparatus 2 may further comprise a switch 24. The switch 24 opens and closes the circuit between the conductive surface 21 and the ground. If the user makes contact with the conductive surface 21 while the switch 24 is open, the charge on the electrical conducting medium 3 will not dissipate to the ground. If the user makes contact with the conductive surface 21 while the switch 24 is closed, the charge on the electrical conducting medium 3 will dissipate to the ground. The switch 24 allows the user to easily create electrical continuity with the ground while keeping contact with the conductive surface 21.

In use, the capacitance article 1 of the present invention is placed on the electrical conducting medium 3 and worn continuously. The attachment portion 12 of the capacitance article 1 in the preferred embodiment is a substantially ring-shaped copper bracelet 13 having a bracelet body 131, a first open end 132, and a second open end 133, with the capacitor 11 attached to the first open end 132. The grounding apparatus 2 in the preferred embodiment is a ball on which the user places their hand, wherein the conductive surface 21 is a metal sphere 211 and the grounding connector 22 is a metal wire 221. At any predetermined interval, the user discharges the built-up charge on the capacitance article 1 by interacting with the grounding apparatus 2 of the present invention. To interact with the grounding apparatus 2 in the preferred embodiment, the user connects the grounding connector 22 to the ground by plugging it into the grounding port of an outlet while the switch 24 is in the open position. In the preferred embodiment, the electrical conducting medium 3 is the body of the user. The user then connects the electrical conducting medium 3 to the conductive surface 21 of the grounding apparatus 2, generally by placing their hand on the conductive surface 21, creating as many points of contact as possible to maximize the surface area of the connection between the electrical conducting medium 3 and the conductive surface 21. Once the electrical conducting medium 3 is placed on the conductive surface 21, the user may interact with the switch 24 to close the circuit. At this point, the user waits briefly for the charge to dissipate from the electrical conducting medium 3 before removing the electrical conducting medium 3 from the conductive surface 21.

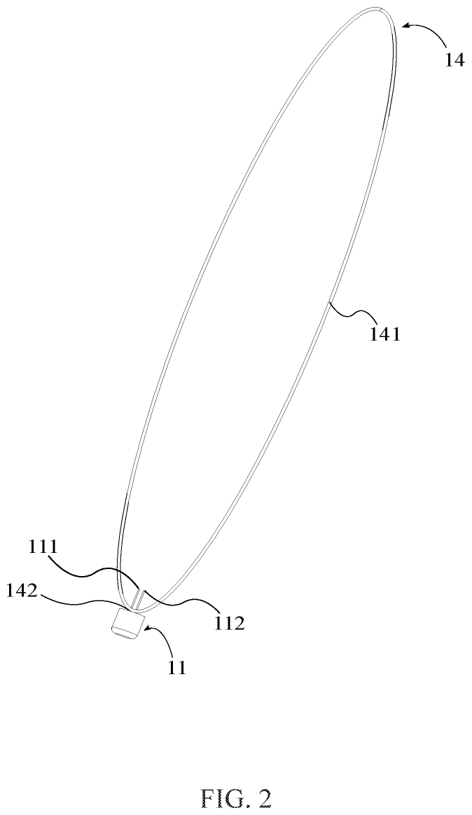

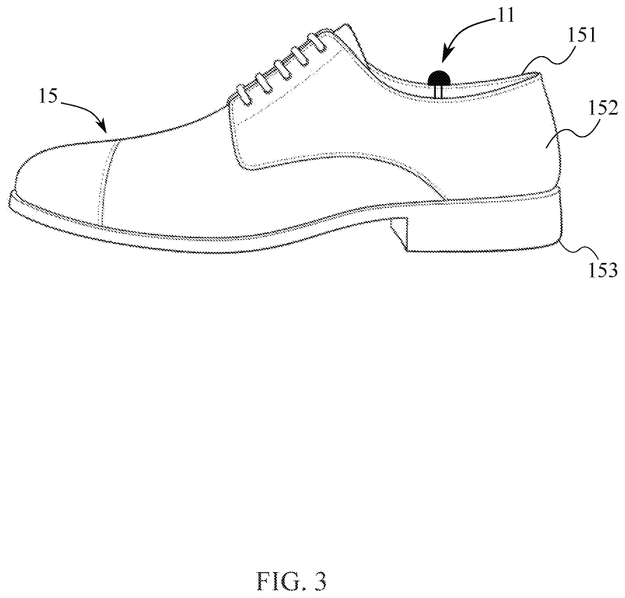

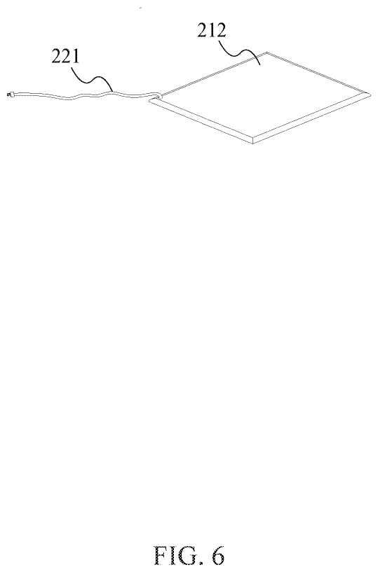

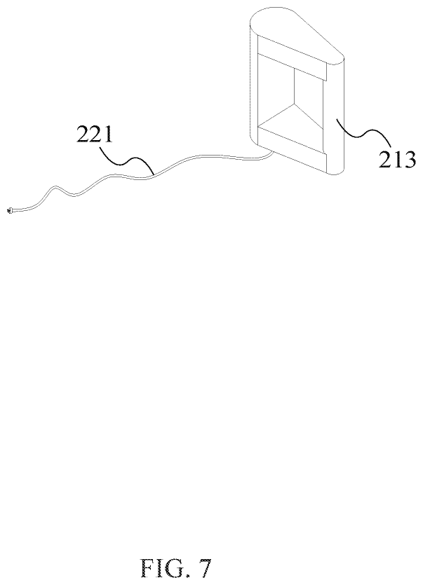

In alternative embodiments of the method of the present invention, alternative capacitance articles 1 and grounding devices may be used. In one embodiment, shown in FIG. 3, the attachment portion 12 of the capacitance article 1 may be a shoe 15 having a topline 151, a shoe body 152, and a sole 153, with the capacitor 11 clipped to the topline 151 of the shoe 15. Alternatively, as shown in FIG. 2, the capacitor 11 may be attached to a closed-loop necklace 14 with a necklace body 141 and an attachment region 142, with the capacitor 11 attached to the attachment region 142. As shown in FIG. 6, the grounding device may be a mat on which the user stands barefoot, wherein the conductive surface 21 is a flat metal plate 212 and the grounding connector 22 is a metal wire 221. Alternatively, as shown in FIG. 7, the grounding apparatus 2 may be a conductive handle which the user grabs with their hand, wherein the conductive surface 21 is a cylindrical metal handle 213 and the grounding connector 22 is a metal wire 221. Any combination of capacitance articles 1 and grounding devices may be used for the method of the present invention.

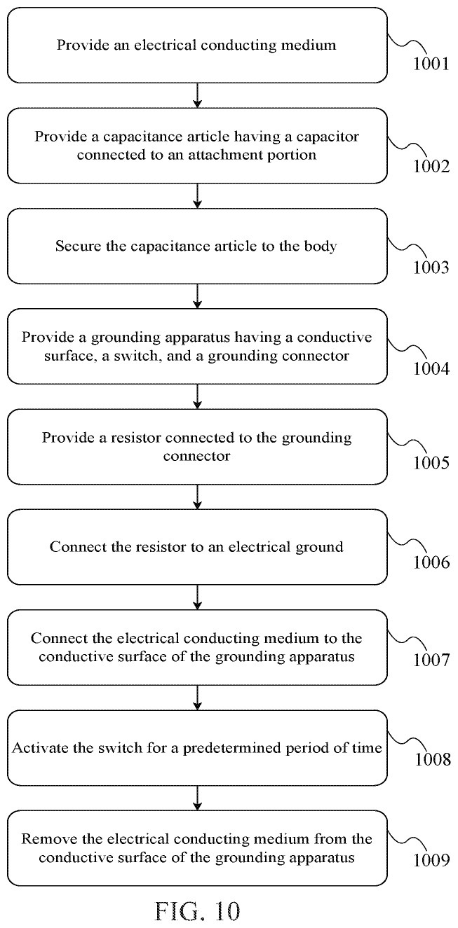

The overall steps of using the system of the present invention are as follows, as described in the flowchart of FIG. 10. In 1001, the electrical conducting medium is provided. in 1002, the capacitance article is provided. In 1003, the capacitance article is secured to the electrical conducting medium, placing the attachment portion in direct physical contact with the electrical conducting medium. in 1004, the grounding apparatus is provided, including the conductive surface, grounding connector, and switch, with the switch operatively connected between the conductive surface and the grounding connector. In 1005, the resistor is then provided and in 1006 connected to the grounding connector, opposite the switch. In 1007, the electrical conducting medium is connected to the conductive surface of the grounding apparatus with multiple points of contact. In 1008, the switch is then activated for a predetermined period of time. Finally, in 1009, the electrical conducting medium is removed from the conductive surface of the grounding apparatus.

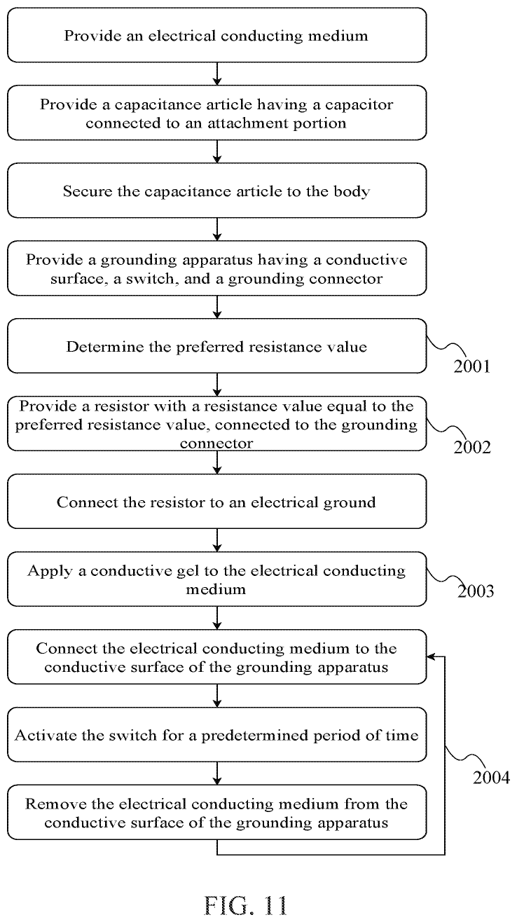

The method of the present invention may include additional steps as shown in the flowchart of FIG. 11. In one embodiment, the step of providing a resistor may further comprise, in 2001, determining the preferred resistance value and, in 2002, selecting a resistor with a resistance value equal to the preferred resistance value. In another embodiment, the method may include, in 2003, the application of a conductive gel or other conductive material between the electrical conducting medium and the grounding apparatus to increase the efficacy of the grounding process. Additionally, the method may include, in 2004, repeating the steps of connecting the electrical conducting medium to the conductive surface through removing the electrical conducting medium from the conductive surface at a predetermined interval. Further, the order of steps of the method of the present invention is not limited to the order as explained in the preferred embodiment and the timing of the steps of the method may vary.

Although the invention has been explained in relation to its preferred embodiment, it is to be understood that many other possible modifications and variations can be made without departing from the spirit and scope of the invention.

What is claimed is:

1. A system of electrical grounding, comprising:
an electrical conducting medium;
a capacitance article;
a grounding apparatus;
an electrical ground;
the capacitance article further comprising a capacitor and an attachment portion;
the grounding apparatus further comprising a conductive surface and a grounding connector;
the capacitor of the capacitance article physically connecting to the attachment portion of the capacitance article;
the attachment portion of the capacitance article physically connecting to the electrical conducting medium;
the conductive surface of the grounding apparatus physically connecting to the grounding connector of the grounding apparatus;
the grounding connector of the grounding apparatus physically connecting to the electrical ground; and
the electrical conducting medium, the capacitance article, and the grounding apparatus configured to electrically connect the capacitor to the electrical ground.

2. The system of electrical grounding of claim 1, further comprising:
a resistor;
the resistor being placed between the grounding connector and the electrical ground; and
the resistor configured to limit the flow of electric current from the grounding connector to the electrical ground.

3. The system of electrical grounding of claim 2, further comprising:
a switch;
the switch being operably connected between the conductive surface and the grounding connector; and
the switch configured to electrically connect the conductive surface to the grounding connector when activated.

4. The system of electrical grounding of claim 3, wherein:
the attachment portion of the capacitance article is a ring-shaped bracelet having a bracelet body, a first open end, and a second open end; and
the capacitor connecting to the bracelet at the first open end.

5. The system of electrical grounding of claim 3, wherein:
the attachment portion of the capacitance article is a closed-loop necklace having a necklace body and an attachment region; and
the capacitor connecting to the necklace at the attachment region.

6. The system of electrical grounding of claim 3, wherein:
the attachment portion of the capacitance article is a shoe having a topline, a shoe body, and a sole;
the capacitor connecting to the shoe at the topline.

7. The system of electrical grounding of claim 3, wherein:
the conductive surface of the grounding apparatus is a metal sphere; and
the grounding connector of the grounding apparatus is a metal wire.

8. The system of electrical grounding of claim 3, wherein:
the conductive surface of the grounding apparatus is flat metal plate; and
the grounding connector of the grounding apparatus is a metal wire.

9. The system of electrical grounding of claim 3, wherein:
the conductive surface of the grounding apparatus is a cylindrical metal handle; and
the grounding connector of the grounding apparatus is a metal wire.

10. A method of storing charge and electrically grounding, comprising:
providing an electrical conducting medium;
providing a capacitance article having a capacitor connected to an attachment portion;
securing the capacitance article to the electrical conducting medium so the attachment portion is in direct physical contact with the electrical conducting medium;
providing a grounding apparatus having a conductive surface, a switch, and a grounding connector; the switch being connected to the conductive surface and the grounding connector and configured to connect the conductive surface to the grounding connector when activated;
providing a resistor connected to the grounding connector, opposite the switch;
connecting the resistor to an electrical ground;
connecting the electrical conducting medium to the conductive surface of the grounding apparatus with more than a single point of contact between the electrical conducting medium and the conductive surface;
activating the switch for a predetermined period of time; and
removing the electrical conducting medium from the conductive surface of the grounding apparatus.

11. The method of storing charge and electrically grounding of claim 10, the step of providing a resistor further comprising:
determining the preferred resistance value; and
selecting a resistor with a resistance value equal to the preferred resistance value.

12. The method of storing charge and electrically grounding of claim 11, wherein:
the preferred resistance value is one megaohm.

13. The method of storing charge and electrically grounding of claim 11, further comprising:
applying a conductive gel to the electrical conducting medium before the step of connecting the electrical conducting medium to the conductive surface of the grounding apparatus.

14. The method of storing charge and electrically grounding of claim 13, further comprising:
repeating the steps of connecting the electrical conducting medium to the conductive surface through removing the electrical conducting medium from the conductive surface at a predetermined interval.

15. The method of storing charge and electrically grounding of claim 10, wherein:
the attachment portion of the capacitance article is a ring-shaped bracelet having a bracelet body, a first open end, and a second open end; and
the capacitor connecting to the bracelet at the first open end.

16. The method of storing charge and electrically grounding of claim 10, wherein:
the attachment portion of the capacitance article 1 is a closed-loop necklace having a necklace body and an attachment region; and
the capacitor connecting to the necklace at the attachment region.

17. The method of storing charge and electrically grounding of claim 10, wherein:
the attachment portion of the capacitance article is a shoe having a topline, a shoe body, and a sole; and
the capacitor connecting to the shoe at the topline.

18. The method of storing charge and electrically grounding of claim 10, wherein:
the conductive surface of the grounding apparatus is a metal sphere; and
the grounding connector of the grounding apparatus is a metal wire.

19. The method of storing charge and electrically grounding of claim 10, wherein:
the conductive surface of the grounding apparatus is flat metal plate; and
the grounding connector of the grounding apparatus is a metal wire.

20. The method of storing charge and electrically grounding of claim 10, wherein:
the conductive surface of the grounding apparatus is a cylindrical metal handle; and
the grounding connector of the grounding apparatus is a metal wire.
